(19) United States
(12) Patent Application Publication
(10) Pub. No.: US 2007/0010602 A1
(43) Pub. Date: Jan. 11, 2007

(54) Nigrosine dye and process for producing the same

(76) Inventors: Tetsuji Kihara, Osaka (JP); Kazuaki Sukata, Osaka (JP)

Correspondence Address: MCGLEW & TUTTLE, PC, P.O. BOX 9227, SCARBOROUGH STATION, SCARBOROUGH, NY, 10510-9227, US

(21) Appl. No.: 10/569,350

(22) PCT Filed: Aug. 26, 2004
(86) PCT No.: PCT/JP04/12297

§ 371 Date: Feb. 22, 2006

(51) Int. Cl. C07D 209/76

(52) U.S. Cl. 524/89; 552/302

(57) ABSTRACT

A non-pyrophoric nigrosine dye and a method of production thereof. A nigrosine dye wherein B−A≦0.04% by weight if, in analysis using a gas chromatograph apparatus equipped with a glass-made sample injection port, the aniline content of the sample determined at a temperature of 200° C. at the sample injection port is written as A% by weight, and the aniline content of the sample determined at a temperature of 260° C. at the sample injection port is written as B% by weight. A method of producing a nigrosine dye which comprises a heat treatment of a neutralized nigrosine base.

### TECHNICAL FIELD

The present invention relates to a non-pyrophoric nigrosine dye and a method of production thereof.

### BACKGROUND ART

In 1867, Coupier prepared nigrosine by acting nitrobenzene on aniline in the presence of hydrochloric acid and iron (Fe) or iron chloride (FeCl₂). Since then, various improvements based on that method have been made (Patent Documents 1 to 5).

Despite these improvements in synthetic methods, the nigrosine dyes obtained had a risk of being pyrophoric, i.e. spontaneous ignition during storage or transportation.

As a method of suppressing this pyrophoric activity, a technique bringing a pyrophoric nigrosine base into contact with an oxygen-containing gas in aniline is disclosed in Patent Document 6 (Patent Document 7). However, the nigrosine dye obtained by the method disclosed in Patent Document 6 (Patent Document 7) was also found to be quite unsatisfactory in terms of conversion to a non-pyrophoric dye.

Patent Document 1: US Patent Publication No. 1896244

Patent Document 2: US Patent Publication No. 1988499

Patent Document 3: US Patent Publication No. 4056530

Patent Document 4: German Patent Publication No. 44406

Patent Document 5: German Patent Publication No. 890104

Patent Document 6: US Patent Publication No. 4359577

Patent Document 7: Japanese Patent Laid-Open No. SHO-56-141354

### DISCLOSURE OF INVENTION

Problems to be Solved by the Invention

The present invention has been done in view of the above-described problems in the prior art, and is directed to provide a non-pyrophoric nigrosine dye and a method of production thereof.

Means of Solving the Problems

The present inventors diligently investigated in an attempt to obtain a non-pyrophoric nigrosine, and found that there is a clear correlation between the aniline content in nigrosine determined by gas chromatography and the pyrophoric activity thereof.

Nigrosine usually contains aniline as an impurity substance. The present inventors took note of the relationship between sample injection port temperature of gas chromatograph apparatus and measured aniline content in the quantitation of the aniline content in nigrosine by gas chromatography (hereinafter also referred to as “the GC method”). When the sample injection port temperature is not higher than a particular temperature, substantially no changes are observed in the relationship between sample injection port temperature and the quantitative value of aniline content in nigrosine, and the aniline occurring as an impurity substance (hereinafter referred to as “impurity aniline”) is quantified nearly as is.

However, when the sample injection port temperature exceeds the aforementioned particular temperature, the quantitative value of aniline content in nigrosine increases gradually with the rise in temperature. This is considered to be due to the aniline liberated by partial decomposition of the nigrosine structure (hereinafter referred to as “free aniline”), and the abundance/scarcity of this free aniline is recognized as being associated with the high/low pyrophoric activity of nigrosine. The free aniline content can be calculated by setting a sample injection port temperature that exceeds the aforementioned particular temperature and is appropriate for the production of a sufficient amount of free aniline, and subtracting the impurity aniline content from the measured aniline content.

In analysis using a gas chromatograph apparatus equipped with a glass-made sample injection port, if the aniline content determined at the sample injection port temperature of 200° C. (corresponding to the impurity aniline content) is written as A% by weight, and the aniline content determined at the sample injection port temperature of 260° C. (corresponding to the sum of impurity aniline and free aniline contents) is written as B% by weight, the pyrophoric activity decreases as the B−A value (corresponding to the free aniline content) decreases; nigrosine dyes wherein B−A≦0.04% by weight are non-pyrophoric. There are no conventional nigrosine dyes wherein B−A≦0.04% by weight.

Accordingly, the nigrosine dye of the present invention is characterized in that B−A≦0.04% by weight if, in analysis using a gas chromatograph apparatus equipped with a glass-made sample injection port, the aniline content of the sample determined at the sample injection port temperature of 200° C. is written as A% by weight, and the aniline content of the sample determined at the sample injection port temperature of 260° C. is written as B% by weight.

A determination of the pyrophoric activity of a nigrosine dye can be conducted according to the specified United Nations recommendations. The above-described nigrosine dye of the present invention does not ignite spontaneously or does not self-heat to 200° C. or higher within 24 hours in a test in which the dye is placed in a cubic container 10 cm in length of each side and kept at a temperature of 140° C. in accordance with recommendations by the United Nations “Recommendations on the TRANSPORT OF DANGEROUS GOODS—Manual of Tests and Criteria—Third revised edition (Class 4, Test method for self-heating substances)”.

The present inventors diligently investigated on the cause(s) of the above-described production of free aniline and the pyrophoric activity in conventional nigrosine dyes, and estimated that the production of free aniline is due to partial decomposition of a nigrosine component, and that the accumulation of heat generated due to partial decomposition of a nigrosine component is a cause of the ignition. The method of the present invention of producing a nigrosine dye is intended to produce a nigrosine dye that permits a reduction in free aniline content due to partial decomposition of a nigrosine component.

Accordingly, the method of the present invention of producing a nigrosine dye is a method of producing the above-described nigrosine dye of the present invention, which comprises a heat treatment of a neutralized nigrosine base.

Because the nigrosine dye of the present invention is non-pyrophoric, it is very safe and hence easy to handle during storage, transportation and the like. Additionally, according to the method of the present invention of producing a nigrosine dye, the non-pyrophoric nigrosine dye of the present invention can easily be prepared from a conventional pyrophoric nigrosine dye.

### BEST MODES OF EMBODIMENT OF THE INVENTION

Any of more specific methods can be used to determine aniline contents for the identification of the nigrosine dye of the present invention as long as the aniline contained in the nigrosine dye can be quantified with high reproducibility at both glass-made sample injection port temperatures of 200° C. and 260° C. As an example, the method used to determine A% by weight and B% by weight (aniline contents determined using a gas chromatograph apparatus at glass-made sample injection port temperatures of 200° C. and 260° C., respectively) in the Examples and Comparative Examples below is described below.

To an accurately weighed 0.1 g sample, 9 ml of a 1:1 mixture of ethanol and chloroform and 1 ml of an internal standard solution (0.1 g of accurately weighed p-nitrotoluene dissolved in a 1:1 [by volume] mixture of ethanol and chloroform, and prepared to obtain a total volume of 100 ml) are added. This mixture is sonicated for 20 minutes and filtered through a filter 0.45 μm in pore diameter, 1 μl (microliter) of the filtrate is injected to the glass-made sample injection port of the following gas chromatograph apparatus, and the aniline content in the sample is quantified.

The gas chromatography apparatus is shown below.

Gas chromatography: HP5890 (manufactured by Hewlett-Packard Company)

Column: DB-1701 (30 m×0.53 mmI.D.×1.0 μm Film), manufactured by J&W Company

Oven: 100° C. to 260° C. (10° C./min)

Injection port: Made of glass, 200° C. or 260° C., split ratio 10:1

Detector: Flame ionization detector (FID), 280° C.

Injection volume: 1 μl

Other columns, for example, DB-5 (manufactured by J&W Company) and the like, may be used instead.

The above-described test for the determination of the pyrophoric activity of a nigrosine dye, i.e., a test in which the dye is placed in a cubic container 10 cm in length of each side and kept at a temperature of 140° C. in accordance with the United Nations recommendations “Recommendations on the TRANSPORT OF DANGEROUS GOODS—Manual of Tests and Criteria- Third revised edition (Class 4, Test method for self-heating substances)”, can specifically be conducted as described below. In the Examples and Comparative Examples below, pyrophoric activity was determined by the following pyrophoric activity test.

A sample is filled in a sample container, which is a cube 10 cm in length of each side made of a stainless steel net having 0.053 mm apertures with its upper portion exposed. The sample-filled sample container is housed in a sample container cover, which is a cube of a size such that the sample container can be accommodated therein, which cube made of a stainless steel net having 0.595 mm apertures with its upper portion exposed. The sample container cover is placed at the center of the inside of a basket 15 cm in length, 15 cm in width and 25 cm in height that is made of a stainless steel net having 0.595 mm apertures, and that is suspended at the center of a thermostatic chamber. Also provided is a thermometer (thermocouple) so that the temperatures at the center of the sample and inside the thermostatic chamber can be measured. After the temperature inside the thermostatic chamber is set at 140° C., this temperature is maintained for 24 hours and sample temperature is continuously recorded for 24 hours. If ignition is observed or sample temperature exceeds 200° C. within 24 hours, the test is discontinued at that time. Time from test initiation to either ignition or the reach of a sample temperature of 200° C. is taken as time to ignition.

The nigrosine dye of the present invention can be produced by, for example, a heat treatment of a pyrophoric nigrosine dye in a solvent. More specifically, the following process can be mentioned.

(1) Heat Treatment of Pyrophoric Nigrosine Dye in the Presence of Organic Acid

The nigrosine dye of the present invention can be prepared by a heat treatment of a pyrophoric nigrosine dye in a solvent using an organic acid as a catalyst.

An organic acid having a pKa of not lower than 0 is preferable, with greater preference given to an organic acid having a pKa of 3 to 5. Particularly preferred is an aliphatic or aromatic carboxylic acid represented by Formula (1) below.
R—(—COOH)ₙ Formula (1)

[In Formula (1), R is an aliphatic group or an aromatic group.]

As specific examples of such carboxylic acids, aliphatic carboxylic acids such as formic acid, oxalic acid, acetic acid, propionic acid, and butyric acid; and aromatic carboxylic acids such as benzoic acid, phthalic acids, benzenetricarboxylic acids, and benzenetetracarboxylic acids can be mentioned.

Using an inorganic acid such as hydrochloric acid or sulfuric acid or a relatively strong organic acid (PKa<0) such as an organic sulfonic acid, it is generally difficult to prepare a non-pyrophoric nigrosine dye wherein B−A≦0.04%.

When an organic acid is used as a catalyst, heat treatment temperature is preferably not lower than 100° C. and not higher than 250° C., more preferably not lower than 160° C. and not higher than 220° C.

Heat treatment time varies depending on the nigrosine used as a raw material, and is generally 5 to 48 hours. When the treatment time is extremely short, no satisfactory effect is obtained in some cases.

(2) Heat Treatment of Pyrophoric Nigrosine Dye in the Presence of Metal Oxide or Heteropolyacid

The nigrosine dye of the present invention can be prepared by a heat treatment of a pyrophoric nigrosine dye in a solvent using a metal oxide or a heteropolyacid as a catalyst.

The aforementioned metal oxide may be one or two or more selected from among silica, alumina, silica alumina, titania, molybdenum oxide (VI), tungsten oxide (VI), and vanadium oxide (V).

Also, the aforementioned heteropolyacid may be one or two or more selected from among H₃PMo₆W₆O₄₀, H₃PW₁₂O₄₀, H₃PMo₁₂O₄₀, H₃PMo₁₀W₂O₄₀, H₃PMo₁₀V₂O₄₀, H₄SiW12O₄₀, and H₄GeMo₁₂O₄₀.

When a metal oxide or a heteropolyacid is used as a catalyst, heat treatment temperature is preferably not lower than 50° C. and not higher than 250° C., more preferably not lower than 100° C. and not higher than 220° C.

Heat treatment time varies depending on the nigrosine used as a raw material, and is generally 5 to 48 hours. When the treatment time is extremely short, no satisfactory effect is obtained in some cases.

(3) Catalyst-Free Heat Treatment of Pyrophoric Nigrosine Dye

The nigrosine dye of the present invention can also be prepared by a catalyst-free heat treatment of a pyrophoric nigrosine dye in a solvent.

In the case of catalyst-free heat treatment, heating temperature is preferably not lower than 200° C. and not higher than 300° C., more preferably not lower than 220° C. and not higher than 250° C.

Heat treatment time varies depending on the nigrosine used as a raw material, and is generally 10 to 60 hours. When the treatment time is extremely short, no satisfactory effect is obtained in some cases.

Although the pyrophoric nigrosine dye (nigrosine) used as the starting material in the above-described processes may be produced by any method, it is desirably a neutralized nigrosine base (specific examples include C.I. Solvent Black 5, C.I. Solvent Black 7, and derivatives thereof). C.I. Solvent Black 7 is more preferable. A nigrosine dye that ignites spontaneously or self-heats to 200° C. or higher within 24 hours in the test in which the sample is placed in a cubic container 10 cm in length of each side and kept at a temperature of 140° C. in accordance with the above-described United Nations recommendations is referred to as a pyrophoric nigrosine dye (nigrosine).

Although all of the above-described processes can be conducted without a solvent, it is preferable to use a solvent from the viewpoint of after-treatment step convenience and the like.

Although various solvents can be used, such as alcoholic solvents, aromatic solvents, aprotic polar solvents, and amine solvents, aromatic amines are particularly suitable.

As examples of such aromatic amines, aniline, methylaniline, dimethylaniline, ethylaniline, diethylaniline, o-toluidine, m-toluidine, p-toluidine, benzylamine, dibenzylamine, tribenzylamine, diphenylamine, triphenylamine, α-naphthylamine, β-naphthylamine and the like can be mentioned. Of these amines, anilines having various substituents or unsubstituted anilines are preferred, with greatest preference given to aniline.

The above-described processes permit heat treatment under pressure.

Also, in all of the above-described processes, treatment temperature can be increased using an autoclave depending on the solvent for use.

### EXAMPLES

The present invention is hereinafter described in more detail by means of the following examples, which, however, are not to be construed as limiting the scope of the invention. In the description below, “%” means “% by weight”.

### Example 1

Ten grams of acetic acid (pKa=4.56) was added to a mixture of 1.0 kg of a pyrophoric nigrosine (B−A=0.11%), which was a commercially available neutralized nigrosine base, and 2.0 kg of aniline, and this solution was refluxed with stirring under atmosphere for 20 hours (the solution temperature during this operation was about 190° C.). Subsequently, the solution was allowed to cool to about 30° C., then twice washed with 1 L (liter) of 2% aqueous solution of NaOH, and further washed with water.

The nigrosine dye obtained was dried under reduced pressure and then subjected to GC analysis; the aniline content A in the case of a sample injection port temperature of 200° C. was 0.21% and the aniline content B in the case of a sample injection port temperature of 260° C. was 0.24%, i.e., B−A=0.03%. This nigrosine dye did not ignite even after 24 hours in the above-described pyrophoric activity test.

### Example 2

When starting materials were treated in the same manner as Example 1, except that reflux treatment time was 30 hours, the aniline content A of the nigrosine dye obtained in the case of a sample injection port temperature of 200° C. was 0.21% and the aniline content B in the case of a sample injection port temperature of 260° C. was 0.22%, i.e., B−A=0.01%. This nigrosine dye did not ignite even after 24 hours in the above-described pyrophoric activity test.

### Example 3

When starting materials were treated in the same manner as Example 1, except that 20 g of propionic acid (pKa=4.67) was used in place of acetic acid, the aniline content A of the nigrosine dye obtained in the case of a sample injection port temperature of 200° C. was 0.18%, and the aniline content B in the case of a sample injection port temperature of 260° C. was 0.20%, i.e., B−A=0.02%. This nigrosine dye did not ignite even after 24 hours in the above-described pyrophoric activity test.

### Example 4

When starting materials were treated in the same manner as Example 1, except that 20 g of benzoic acid (pKa=4.00) was used in place of acetic acid, the aniline content A of the nigrosine dye obtained in the case of a sample injection port temperature of 200° C. was 0.15%, and the aniline content B in the case of a sample injection port temperature of 260° C. was 0.18%, i.e., B−A=0.03%. This nigrosine dye did not ignite even after 24 hours in the above-described pyrophoric activity test.

### Example 5

When starting materials were treated in the same manner as Example 1, except that 40 g of silica gel for column chromatography (manufactured by Wako Pure Chemical Industries) was used in place of acetic acid and that silica gel was removed by filtration after refluxing, the aniline content A of the nigrosine dye obtained in the case of a sample injection port temperature of 200° C. was 0.52%, and the aniline content B in the case of a sample injection port temperature of 260° C. was 0.54%, i.e., B−A=0.02%. This nigrosine dye did not ignite even after 24 hours in the above-described pyrophoric activity test.

### Example 6

When starting materials were treated in the same manner as Example 5, except that reflux treatment time was 24 hours, the aniline content A of the nigrosine dye obtained in the case of a sample injection port temperature of 200° C. was 0.63% and the aniline content B in the case of a sample injection port temperature of 260° C. was 0.64%, i.e., B−A=0.01%. This nigrosine dye did not ignite even after 24 hours in the above-described pyrophoric activity test.

### Example 7

When starting materials were treated in the same manner as Example 5, except that 40 g of Molecular Sieves 4A (silica alumina manufactured by Aldrich Company) was used in place of silica gel, the aniline content A of the nigrosine dye obtained in the case of a sample injection port temperature of 200° C. was 0.31% and the aniline content B in the case of a sample injection port temperature of 260° C. was 0.33%, i.e., B−A=0.02%. This nigrosine dye did not ignite even after 24 hours in the above-described pyrophoric activity test.

### Example 8

When starting materials were treated in the same manner as Example 6, except that 30 g of H₃PMo₆W₆O₄₀ was used in place of silica gel, the aniline content A of the nigrosine dye obtained in the case of a sample injection port temperature of 200° C. was 0.11% and the aniline content B in the case of a sample injection port temperature of 260° C. was 0.12%, i.e., B−A=0.01%. This nigrosine dye did not ignite even after 24 hours in the above-described pyrophoric activity test.

### Example 9

When starting materials were treated in the same manner as Example 5, except that 40 g of H₃PMo₁₀V₂O₄₀ was used in place of silica gel, the aniline content A of the nigrosine dye obtained in the case of a sample injection port temperature of 200° C. was 0.11% and the aniline content B in the case of a sample injection port temperature of 260° C. was 0.13%, i.e., B−A=0.02%. This nigrosine dye did not ignite even after 24 hours in the above-described pyrophoric activity test.

### Example 10

A mixture of 1.0 kg of a pyrophoric nigrosine (B−A=0.11%) and 1.0 kg of aniline was heated at 220° C. with stirring in an autoclave for 48 hours.

The nigrosine dye obtained was dried under reduced pressure and then subjected to GC analysis; the aniline content A in the case of a GC sample injection port temperature of 200° C. was 0.21% and the aniline content B in the case of a sample injection port temperature of 260° C. was 0.25%, i.e., B−A=0.04%. This nigrosine dye did not ignite even after 24 hours in the above-described pyrophoric activity test.

### Comparative Example 1 (Example 1 shown in Patent Documents 6 and 7)

The mixture of 1.0 kg of a pyrophoric nigrosine and 2.0 kg of aniline, used in Example 1, was sparged with air (500 ml/min) at 75° C. during vigorous stirring for 16 hours. Subsequently, this solution was dried until the total content of volatile substances such as aniline became not higher than 1%, and then cooled.

The product obtained was subjected to GC analysis; the aniline content A in the case of a GC sample injection port temperature of 200° C. was 0.51% and the aniline content B in the case of a sample injection port temperature of 260° C. was 0.63%, i.e., B−A=0.12%. It ignited after 6.6 hours when tested using the above-described pyrophoric activity test method, although Patent Documents 6 and 7 state that the samples obtained were non-pyrophoric.

### Comparative Example 2 (Example 2 shown in Patent Documents 6 and 7)

The mixture of 1.0 kg of a pyrophoric nigrosine and 2.0 kg of aniline, used in Example 1, was sparged with air (16 l/min [liter/min]) at 50 to 55° C. during vigorous stirring for 20 hours. Subsequently, this solution was dried until the total content of volatile substances such as aniline became not higher than 1%, and then cooled.

The product obtained was subjected to GC analysis; the aniline content A in the case of a GC sample injection port temperature of 200° C. was 0.44% and the aniline content B in the case of a sample injection port temperature of 260° C. was 0.53%, i.e., B−A=0.09%. It ignited after 7.0 hours when tested using the above-described pyrophoric activity test method, although Patent Documents 6 and 7 state that the samples obtained were non-pyrophoric.

### Comparative Example 3

When starting materials were treated in the same manner as Example 1, except that 10 g of hydrochloric acid (pKa=−7.0) was used in place of acetic acid, the aniline content A of the product obtained in the case of a sample injection port temperature of 200° C. was 0.47% and the aniline content B in the case of a sample injection port temperature of 260° C. was 0.78%, i.e., B−A=0.31%. This sample ignited after 3.5 hours.

### Comparative Example 4

When starting materials were treated in the same manner as Example 1, except that 20 g of toluenesulfonic acid (pKa=−6.5) was used in place of acetic acid, the aniline content A of the product obtained in the case of a sample injection port temperature of 200° C. was 0.57% and the aniline content B in the case of a sample injection port temperature of 260° C. was 0.83%, i.e., B−A=0.26%. This sample ignited after 3.2 hours.

### Comparative Example 5

When starting materials were treated in the same manner as Example 1, except that reflux treatment time was extremely shortened (3 hours), the aniline content A of the product obtained in the case of a sample injection port temperature of 200° C. was 0.20% and the aniline content B in the case of a sample injection port temperature of 260° C. was 0.26%, i.e., B−A=0.06%. This sample ignited after 13.5 hours.

### Comparative Example 6

When starting materials were treated in the same manner as Example 5, except that reflux treatment time was extremely shortened (3 hours), the aniline content A of the product obtained in the case of a sample injection port temperature of 200° C. was 0.19% and the aniline content B in the case of a sample injection port temperature of 260° C. was 0.30%, i.e., B−A=0.11%. This sample ignited after 7.0 hours.

The results of the Examples and Comparative Examples are summarized in Table 1.

TABLE 1

| | | B − A | Time (hr) to |
|---|---|---|---|
| | Treatment conditions | (%) | ignition |
| Example 1 | Acetic acid (organic acid) | 0.03 | >24 |
| | 20 hours | | |
| Example 2 | Acetic acid (organic acid) | 0.01 | >24 |
| | 30 hours | | |
| Example 3 | Propionic acid (organic acid) | 0.02 | >24 |
| | 20 hours | | |
| Example 4 | Benzoic acid (organic acid) | 0.03 | >24 |
| | 20 hours | | |
| Example 5 | Silica gel (metal oxide) | 0.02 | >24 |
| | 20 hours | | |
| Example 6 | Silica gel (metal oxide) | 0.01 | >24 |
| | 24 hours | | |
| Example 7 | Molecular Sieves 4A (metal oxide) | 0.02 | >24 |
| | 20 hours | | |
| Example 8 | H₃PMo₆W₆O₄₀ | 0.01 | >24 |
| | (heteropolyacid) 24 hours | | |
| Example 9 | H₃PMo₁₀V₂O₄₀ | 0.02 | >24 |
| | (heteropolyacid) 20 hours | | |
| Example 10 | Autoclave (catalyst-free) | 0.04 | >24 |
| | 48 hours | | |
| Comparative | Air sparging (75° C.) | 0.12 | 6.6 |
| Example 1 | 16 hours | | |
| Comparative | Air sparging (55° C.) | 0.09 | 7.0 |
| Example 2 | 20 hours | | |
| Comparative | Hydrochloric acid (pKa = −7.0) | 0.31 | 3.5 |
| Example 3 | 20 hours | | |
| Comparative | Toluenesulfonic acid | 0.26 | 3.2 |
| Example 4 | (pKa = −6.5) 20 hours | | |
| Comparative | Acetic acid (organic acid) | 0.06 | 13.5 |
| Example 5 | 3 hours | | |
| Comparative | Silica gel (metal oxide) | 0.11 | 7.0 |
| Example 6 | 3 hours | | |

1. A nigrosine dye characterized in that B−A≦0.04% by weight if, in analysis using a gas chromatograph apparatus equipped with a glass-made sample injection port, the aniline content thereof determined at the aforementioned sample injection port temperature of 200° C. is written as A% by weight and the aniline content thereof determined at the aforementioned sample injection port temperature of 260° C. is written as B% by weight.

2. The nigrosine dye of claim 1, which does not ignite spontaneously or does not self-heat to 200° C. or higher within 24 hours in a test in which the dye is placed in a cubic container 10 cm in length of each side and kept at a temperature of 140° C. in accordance with the United Nations recommendations “Recommendations on the TRANSPORT OF DANGEROUS GOODS—Manual of Tests and Criteria—Third revised edition (Class 4, Test method for self-heating substances)”.

3. The nigrosine dye of claim 1, which is prepared by a heat treatment of a neutralized nigrosine base.

4. The nigrosine dye of claim 1, which is prepared by a heat treatment of a neutralized nigrosine base in various solvents in the presence of an organic acid as a catalyst.

5. The nigrosine dye of claim 1, which is prepared by a heat treatment of a neutralized nigrosine base in various solvents in the presence of a metal oxide as a catalyst.

6. The nigrosine dye of claim 1, which is prepared by a heat treatment of a neutralized nigrosine base in various solvents in the presence of a heteropolyacid as a catalyst.

7. The nigrosine dye of claim 1, which is prepared by a heat treatment of a neutralized nigrosine base in various solvents without a catalyst.

8. The nigrosine dye of claim 4, said solvent being anilines.

9. The nigrosine dye of claim 4, said solvent being aniline.

10. The nigrosine dye of claim 4, wherein the pKa of said organic acid is not lower than 0.

11. The nigrosine dye of claim 4, wherein the pKa of said organic acid is 3 to 5.

12. The nigrosine dye of claim 11, wherein said organic acid is an aliphatic or aromatic carboxylic acid represented by Formula (1) below,
R—(—COOH)ₙ Formula (1),
in Formula (1), R being an aliphatic group or an aromatic group.

13. The nigrosine dye of claim 5, wherein said metal oxide is one or two or more selected from among silica, alumina, silica alumina, titania, molybdenum oxide (VI), tungsten oxide (VI), and vanadium oxide (V).

14. The nigrosine dye of claim 6, wherein said heteropolyacid is one or two or more selected from among H₃PMo₆W₆O₄₀, H₃PW₁₂O₄₀, H₃PMo₁₂O₄₀, H₃PMo₁₀W₂O₄₀, H₃PMo₁₀V₂O₄₀, H₄SiW₁₂O₄₀, and H₄GeMo₁₂O₄₀.

15. The nigrosine dye of claim 4, wherein the temperature for said heat treatment is not lower than 100 and not higher than 250.

16. The nigrosine dye of claim 5, wherein the temperature for said heat treatment is not lower than 50 and not higher than 250.

17. The nigrosine dye of claim 7, wherein the temperature for said heat treatment is not lower than 200 and not higher than 300.

18. The nigrosine dye of claim 3, wherein said heat treatment is conducted under pressure.

19. A method of producing the nigrosine dye of claim 1, which comprises a heat treatment of a neutralized nigrosine base.

20. The method of producing a nigrosine dye of claim 19, wherein said heat treatment is conducted in a solvent.

21. The method of producing a nigrosine dye of claim 19, wherein said heat treatment is conducted in the presence of an organic acid, a metal oxide, or a heteropolyacid as a catalyst.

22. The method of producing a nigrosine dye of claim 19, wherein said heat treatment is conducted under pressure.

23. The method of producing a nigrosine dye of claim 19, said solvent being aniline.
